(19) United States
(12) Patent Application Publication
(10) Pub. No.: US 2019/0083531 A1
(43) Pub. Date: Mar. 21, 2019

(54) PURIFIED BLOOD FOR USE IN CANCER THERAPY

(71) Applicant: Bavarian Immunology Association GmbH, Prien am Chiemsee (DE)

(72) Inventors: Meredith Rigdon Lentz, Chieming (DE); Kiran Lentz, Chieming (DE)

(73) Assignee: Bavarian Immunology Association GmbH, Prien am Chiemsee (DE)

(21) Appl. No.: 16/128,777

(22) Filed: Sep. 12, 2018

(30) Foreign Application Priority Data
Sep. 13, 2017 (EP) 17001529.1

(51) Int. Cl. A61K 35/14; A61P 35/04; A61M 1/36

(52) U.S. Cl. CPC A61K 35/14; A61M 2202/0415; A61M 1/362; A61P 35/04

(57) ABSTRACT

The present invention is generally in the field of enhancing an immune response. Particularly, it relates to purified blood for use in a method of treating cancer in a patient by extracorporeally removing inhibitors of immune mediators.

### PRIORITY DATA

This application claims priority to European Patent Application No. EP 17001529.1, filed Sep. 13, 2017, which is incorporated herein by reference in its entirety.

### BACKGROUND

The present invention is generally in the field of enhancing an immune response. Particularly, it relates to purified blood for use in a method of treating cancer in a patient by extracorporeally removing inhibitors of immune mediators.

Conventional cancer therapy is based on the use of drugs and/or radiation which kills replicating cells, hopefully faster than the agents kill the patient's normal cells. Surgery is used to reduce tumor bulk, but has little impact once the cancer has metastasized. Radiation is effective only in a localized area. The treatments can in themselves kill the patient, in the absence of maintenance therapy. For example, for some types of cancer, bone marrow transplants have been used to maintain the patient following treatment with otherwise fatal amounts of chemotherapy. “Cocktails” of different chemotherapeutic agents and combinations of very high doses of chemotherapy with restorative agents, for example, granulocyte macrophage colony stimulating factor (“GM-CSF”), erythropoietin, thrombopoetin granulocyte stimulating factor, (“G-CSF”), macrophage colony stimulating factor (“M-CSF”) and stem cell factor (“SCF”) to restore platelet and white cell levels, have been used to treat aggressive cancers. Even with the supportive or restrictive therapy, side effects are severe.

WO 2001/037873 describes an alternative method for treating cancer, involving ultrapheresis to remove compounds based on molecular weight, which promotes an immune attack on the tumors by the patient's own white cells.

Despite all of these efforts, many patients die from cancer; others are terribly mutilated.

It is therefore an object of the present invention to provide compositions for use in methods for the treatment of cancer.

Surprisingly, it was found that cancer patient may be extracorporeal treated by continuously removing inhibitors of immune mediators, namely soluble TNF receptor-1 (TNF-R1) and soluble TNF receptor-2 (TNF-2) and optionally further inhibitors of immune mediators, from the patient's blood.

Accordingly, the present invention relates to purified blood for use in a method of treating cancer in a patient, the method comprising an extracorporeal blood purification process comprising
a) including an extracorporeal circuit comprising a blood-fractionating device having a plasma-separation element and an immune adsorption element into the patient's blood circle;
b) passing the patient's blood through the extracorporeal circuit, wherein
   (i) blood is continuously taken from the patient;
   (ii) the blood is separated into blood plasma and remainder, wherein blood is passed through the plasma-separation element with a flow rate of at least 300 cm³/min;
   (iii) the blood plasma is purified from soluble TNF receptor-1 (TNF-R1) and soluble TNF receptor-2 (TNF-2) in the immune adsorption element, wherein a plasma volume corresponding to at least 20% of the patient's weight is passed through the immune adsorption element per day;
   (iv) blood is restored by combining the purified blood plasma and the remainder; and
   (v) the restored blood is continuously returned into the patient; and
c) separating the extracorporeal circuit from the patient's blood circle, wherein the blood purification process is performed on at least six days within a period of at most four weeks.

The efficient and practicable removal of inhibitors of immune mediators, namely soluble TNF receptor-1 (TNF-R1) and soluble TNF receptor-2 (TNF-2) and optionally further inhibitors of immune mediators, from the patient's blood is decisive in the present invention.

TNF-receptor 1 (TNF-R1, TNF-BP1, Type B, 55 kD or HTR antigen) and TNF-receptor 2 (TNF-R2, TNF-BP II, Type A, 75 kD or UTR antigen) are proteins with molecular weights of 55 and 75 kD respectively. TNF-R1 is expressed in most tissues, and can be fully activated by both the membrane-bound and soluble trimeric forms of TNF, whereas TNF-R2 is found only in cells of the immune system, and responds to the membrane-bound form of the TNF homotrimer. Both TNF-R1 and TNF-R2 are “death receptor” (DR) pathways, but their cytotoxic effects are triggered via different intracellular mechanisms. Binding of the ligand TNF to TNF-R1 appears to cause very rapid depletion of intracellular anti-oxidants and death by oxidative stress. Binding of TNF to TNF-R2 causes downstream signaling that culminates in activation of the executioner caspases, caspase-3, caspase-7 and caspase-9, resulting in apoptosis.

Cancer patients show high concentrations of the sTNF-R1 and sTNF-R2—and optionally receptor types of a few other immune mediators—in body fluids, apparently originating in the tumor micro-environment. Soluble TNF-receptors (sTNF-Rs) are truncated versions of membrane TNF-Rs, consisting of only the extracellular binding domain (the ectodomain). Although sTNF-Rs necessarily lose their signaling capacity given that they are disengaged from the cell surface, they maintain full binding capacity. sTNF-Rs intercept the TNF/LT cytokines before they can bind with tumor cell surface receptors, thus neutralizing TNF/LT in the tumor micro-environment.

The basis of the treatment according to the present invention is to employ apheresis coupled with an affinity column containing a proprietary biologic to remove inhibitory receptors from patient's plasma in a controlled manner, thereby disrupting immunoevasion in the tumor microenvironment and inducing controlled tumor inflammation and necrosis. The treatment is purely subtractive and highly specific, resulting in the removal of target inhibitors and nothing else.

Clinically, the inventors have observed that the rate of tumor destruction is a function of the level to which the immune inhibitors are reduced in the patient's plasma and the duration for which the reduced levels of these inhibitors are maintained.

Accordingly, in the present invention purified blood is used in a method of treating cancer in a patient. The term “purified blood” relates to blood which has been purified from sTNF-R1 and sTNF-R2—and optionally receptor types of a few other immune mediators. In accordance with the present invention the blood has been purified by immune adsorption, which specifically removes sTNF-R1 and sTNF-R2 (and optionally receptor types of other immune mediators, if intended), but maintains other blood components. The removal is in a process which is referred to as blood purification process.

In a first step of the process, an extracorporeal circuit comprising a blood-fractionating device having a plasma-separation element and an immune adsorption element is included into the patient's blood circle. The extracorporeal circuit relates to an apparatus, which takes blood from the patient's circulation, carries it outside the body and optionally treats it and returns it into the patient's circulation.

For this, the patient will typically be connected to the apparatus using an indwelling venous catheter and standard intravenous tubing, with connections similar to those used for other extracorporeal blood treatment systems, so that blood can be removed from and returned to the patient. An exemplary device is described in EP 1 949 915.

As a second step, the patient's blood is passed through the extracorporeal circuit. For this, the blood is continuously taken from the patient and transported to the blood-fractionating device. Typically, the device is first flushed with saline and then treated with an anticoagulant or anticlotting agent, such as sodium heparin or anticoagulant citrate dextrose, to be sure that there are no locations within the system where blood clotting can occur. Moreover, small amounts of anticoagulants may be continually introduced into the blood stream directed to the device to ensure than no clotting occurs during the filtration process. All of the surfaces of the system which come in contact with the blood and fluids which are infused into the patient must be either sterilized or prepared aseptically prior to commencing treatment.

The treatment of the blood occurs in the blood-fractionating device comprising a plasma-separation element and an immune adsorption element. Although it is possible to treat whole blood to remove soluble cytokine receptor inhibitors, it has been found advantageous to first separate formed elements (blood cells and larger proteins) and a blood fraction referred to as plasma and treat/purify the plasma. This provides for fewer potential problems due to damage to the red cells or activation of the white cells as they pass through the column or filter for removal of the inhibitors. Systems for separating blood into the cellular and other larger components and plasma (here referred to as plasma-separation elements) are commercially available. A suitable system is the B. Braun Diapact CCRT plasma exchange/plasma profusion controller with plasma profusion tubing. Other systems include the Fresenius Hemocare Apheresis system, the Gambo Prisma System and the Asahi and Kurray blood filtration controllers and the Exorim Immuoadsorption Systems. The blood is separated into blood plasma and remainder, wherein blood is passed through the plasma-separation element with a flow rate of at least 300 cm³/min. The high flow rate allows to purify the blood plasma to the required extend in an acceptable amount of time. Typically, a blood pump is used to control the flow rate. Preferably, the plasma-separation element has a sieving coefficient of at least 90%, preferably at least 95%, more preferably at least 99% for albumin and/or a sieving coefficient of at most 10%, preferably at most 5%, more preferably at most 1% for fibrinogen. Still more preferably, the plasma-separation element has a sieving coefficient of at least 99% for albumin and a sieving coefficient of at most 1% for fibrinogen. Accordingly, albumin will essentially be maintained in plasma, whereas fibrinogen is essentially retained in the remainder.

After passing the blood through the plasma-separation element, the plasma is directed to the immune adsorption element, where the blood plasma is purified from soluble TNF receptor-1 (TNF-R1) and soluble TNF receptor-2 (TNF-2), wherein a plasma volume corresponding to at least 20% of the patient's weight is passed through the immune adsorption element per day.

Selective removal of the soluble TNF-R1 and TNF-R2 (which function as inhibitors of the cytokine) can be used to promote a selective, safe inflammatory response against a tumor. The binding agent may be an antibody reactive with the receptor, its naturally occurring ligand TNF or a mutant, fragment or epitope thereof still capable of selectively binding to the receptor. As used herein, the term “selectively binds” means that a molecule binds to one type of target molecule, but not substantially to other types of molecules. The term “specifically binds” is used interchangeably herein with “selectively binds”. As used herein, the term “binding partner” or “binding agent” is intended to include any molecule chosen for its ability to selectively bind to the targeted immune system inhibitor. The binding partner can be one which naturally binds the targeted immune system inhibitor. For example, tumor necrosis factor alpha or beta can be used as a binding partner for sTNF-R. Alternatively, other binding partners, chosen for their ability to selectively bind to the targeted immune system inhibitor, can be used. These include fragments of the natural binding partner, polyclonal or monoclonal antibody preparations or fragments thereof, or synthetic peptides. Antibodies to the receptor proteins can be generated by standard techniques, using human receptor proteins. Alternative, the naturally occurring ligand to the receptor may be used or a mutant, fragment or epitope thereof, which is based on the ligand, but has been modified e.g. in order to increase stability, binding affinity or to confer any other biological, chemical or physical characteristics.

The receptors are removed from the plasma by binding them to the binding agent. The binding agents can be immobilized on a filter, in a column, or using other standard techniques for binding reactions to remove proteins from the blood or plasma of a patient. As used herein, antibody refers to antibody, or antibody fragments (single chain, recombinant, or humanized), immunoreactive against the receptor molecules. In the most preferred embodiment, the antibody is reactive with the carboxy-terminus of the shed receptor molecules, thereby avoid concerns with signal transduction by the receptor is still present on the cell surface. Antibodies can be obtained from various commercial sources such as Genzyme Pharmaceuticals. These are preferably humanized for direct administration to a human, but may be of animal origin if immobilized in an extracorporeal device. The binding agent and immune adsorption element should be sterilized and treated to remove endotoxin and other materials not acceptable for administration to a patient.

In the preferred embodiment, the binding agent is immobilized on a solid support, such as a SEPHAROSE™ column, using standard techniques such as cyanogen bromide or commercially available kits for coupling of proteins to supports formed of materials such as nitrocellulose or polycarbonate. In the preferred embodiment, plasma is circulated through an inert polymeric matrix, such as SEPHAROSE™ sold by Amersham-Biosciences, Upsala, Sweden, within a medical grade polycarbonate housing approximately 325 ml in volume, supplied by Tacoma Plastics, as shown in FIG. 1. Other equivalent materials can be used. These should be sterilizable or produced aseptically and be suitable for connection using standard apheresis tubing sets. Typical materials include acrylamide and agarose particles or beads. Other suitable matrices are available, and can be formed of acrylamide or other inert polymeric material to which antibody can be bound. Standard techniques for coupling of antibodies to the gel material are used.

In another embodiment, the binding partners are immobilized to filter membranes or capillary dialysis tubing, where the plasma passes adjacent to, or through, the membranes to which the binding partners are bound. Moreover, the binding agent may be bound to particles that are exposed to the blood or plasma within a mesh or reactor having retaining means.

However, columns are preferred and may be produced as detailed in the following exemplary description: The immobilizing binding agent/partner is packed into the column after sterilization or aseptic treatment of the material. Coupling to the matrix using a technique such as cyanogen bromide significantly reduces virus due either to removal of the unbound virus during washing or by coupling the virus to the matrix material, which inactivates the bound virus. Due to recent concerns regarding the potential for viruses from the animals used to make polyclonal antibodies, such as the rabbits used to make the antibodies in the following examples, the antibody is bound to the matrix material, the matrix material is placed into a bag which is then spread to provide for maximum exposed surface area and treated by stationary e-beam radiation (24 centi). This can cause up to 25% loss of activity and antibody quantities may have to be increased accordingly. Other known sterilization techniques that may be used, alone or in combination, include washing the matrix material containing immobilized binding partner with glycine at a pH of 2.8 which destroys enveloped virus (two to three log reduction); ultraviolet irradiation which causes a four to five log reduction of all viruses with only about 5% loss of antibody activity. The sterilized or aseptically prepared matrix material is transferred from the bag through a sterile port in the bag directly into the sterilized column port. Column housings are sterilized prior to packing with immobilized antibody, which is done using aseptic conditions. Columns are filled with 0.1% sodium azide in phosphate buffered saline (“PBS”) as a preservative, although other medically equivalent buffers could be used. These are stored refrigerated until use. Columns may be regenerated by washing with normal sterile saline, elution with 200 mM glycine-HCl pH 2.8, washing with normal sterile saline, then washing with PBS. Other equivalent washing solutions can be used. The column is flushed with multiple volumes of sterile saline prior to use.

The immunopheresis column IAC122 is a sterile immune adsorbent product designed to remove soluble inhibitors to pro-inflammatory cytokines from the blood. It is designed to be used in conjunction with commercially available approved extracorporeal blood treatment systems. (e.g. Diapact CRRT device, B. Braun, Fresenius Hemocare Apheresis, Exorim Immuoadsorption Systems.). The device is intended only to be sold on the order of and used only by physicians with experience in the use of immunoadsorption techniques. The immune adsorption column is intended to remove soluble pro-inflammatory cytokines which are known to be overproduced in certain disease states like cancers, where they are a major cause of immune tolerance of tumor associated neo-antigen. In clinical application in cancer patients the removal of these inhibitors/shed receptors may produce tumor specific inflammation which can lead to tumor destruction. The column housing is a 325 ml volume medical grade polycarbonate device (PNS-400146-Fresenius HemoCare, INC). The column matrix is composed of Sephrose 4B beads and a binding agent for against pro-inflammatory cytokine inhibitors (soluble receptors to tumor necrosis factor alpha (TNF) and interleukine 2 (IL2)). Therefore, the essential components for manufacturing are Sepharose, purchased as sterile product from Amersham-Biosciences (Upsala, Sweden), antibodies to TNF receptors and IL2 receptor that are sterilized by filtration (Eurogentec, Liege, Belgium), and a polycarbonate housing (Fresenius, St. Walin), sterilized by autoclave. Sterile components and aseptic technique during the production, as well as final product testing of each column or column production lot are central to the safety of this medicinal device product. Each column is constructed under aseptic conditions according to the GMP. Each column is individually tested for sterility and endotoxin level post manufacture. Each column is filled with 0.1% Sodium Azide (NaAzide) in PBS and maintained between 4-8° C. prior to clinical use. The intended purpose of the device is to serve as an adsorption column in clinical apheresis procedures. The column is part of an extracorporeal circuit using a standard plasma perfusion machine that removes blood from patients, separates the plasma by filtration, passes the filtered plasma through an adsorption column and then return the combined plasma and cell fractions to the patient in a continuous loop system. The adsorptive material in the column is constructed to specifically bind two kinds of soluble receptors to Tumor Necrosis Factor α (sTNF-R1 and sTNF-R2) and also to bind soluble receptors to interleukine 2 (sIL2R). The goal of using these columns in apheresis procedures is to remove those inhibitors from the blood that are known to protect tumor cells against destruction by the host immune system.

After plasma has been purified, blood is restored by combining the purified blood plasma and the remainder and the restored blood is continuously returned into the patient. This may be done by using either a single catheter site or a second site. A venous air trap may be used for combining plasma and remainder, where it is mixed with patient's blood. Standard microprocessor controls can be used to regulate the blood flow, for example, by monitoring the volume of the blood products being removed, in combination with flow rate monitors and pump speed.

As a third step, the extracorporeal circuit is separated from the patient's blood circle as known by the person skilled in the art.

Treatment is conducted over a period of time until a positive indication is observed. This is typically based on diagnostic tests which show that there has been some reduction in tumor size or which suggests tumor inflammation. The patient is preferably treated until diagnostic tests conducted verity that there has been shrinkage of the tumors and/or inflammation. Then, treatment regime is continued for some time to maintain the disease state and to stabilize the patient.

For an effective treatment, the blood purification process is performed on at least six days within a period of at most four weeks in order to keep the concentrations of the inhibitors, namely sTNF-R1 and sTNF-R2, in the patient's blood at a low level.

The patient is usually treated for a period of time sufficient to lower the levels of circulating sTNF-R1 and sTNF-R2. Treatment cycles typically consist of three or more treatments per week and/or a total of twelve or more treatments, over a period of time for up to five weeks. Treatment cycles can be repeated as required. Typically, a patient is treated every day from Monday to Friday for at least three weeks. Diagnostic tests may be conducted to verify that there has been shrinkage of the tumors, and then the treatment regime is repeated as needed.

Preferably, the treatment frequency and duration is chosen to reduce the levels to at least 5% less than normal values (healthy control subject or cohort); in another embodiment, the levels are reduced to at least 10% less than normal values. Circulating levels of the inhibitors frequently rise significantly following treatment, which may be due to shedding by the tumors. In the preferred embodiment, the plasma is treated so that normal levels of circulating inhibitors are achieved within the first hour of treatment. Treatment is then continued so that levels are reduced below normal and maintained at less than normal levels for a period of at least four to five hours. However, the degree of reduction in the levels of the inhibitors must be balanced by the type of tumor to be treated and the tumor burden. Lowering the concentration of these receptors induces an inflammatory response against the tumor cells. Evidence of an inflammatory response includes fever, tumor specific inflammatory pain, tumor swelling and tumor necrosis. Other problems that can occur include tumor lysis syndrome, which can be treated with standard medical management by qualified physicians.

In a preferred embodiment, the blood purification process is performed once, twice, three times or four times a day, until the intended daily plasma volume has been purified by passing it through the immune adsorption element. It can be desirable to perform several purification processes with the patient at one day. The time needed for the purification of the intended plasma volume might be found to long for a single session. The patient might need time to eat, move, or recover etc. after some time of treatment. If so, the purification process may be interrupted and continue or newly started after a break. Depending on the patient's needs, one or more purification processes may be performed on every treatment day.

In another embodiment, the blood purification process is performed on at least 6 days, preferably at least 8 days, more preferably at least 10 days, within a period of at most 21 weeks, preferably within a period of at most 15 days.

In a preferred embodiment, the method is continued for at least one month, at least six weeks, at least two months or at least three months. Evidently, the frequency and duration of treatment may depend on the patient's need (level of inhibitors of immune mediators, clinical status, availability etc.), the clinical routine in the medical facility and other factors. However, the skilled practitioner will be able to choose suitable parameters in accordance with the prevailing circumstances.

Due to the high flow rates used in the present blood purification process, it is possible to pass high amounts of blood and plasma through the extracorporeal circuit. Accordingly, in preferred embodiment, a plasma volume corresponding to at least 25%, preferably 30%, of the patient's weight is passed through the immune adsorption element per day and/or at least 30 l, preferably at least 40 l, more preferably at least 50 l, of the patient's blood are passed through the extracorporeal circuit per day. Moreover, in a preferred embodiment, blood is passed through the plasma-separation element with a flow rate of at least 350 cm³/min.

In another preferred embodiment, the immune adsorption element comprises a column, on which binding agents for sTNF-R1 and sTNF-R2 are covalently bound.

In still another preferred embodiment, the blood plasma is further purified from at least one further inhibitor of an immune mediator, particularly an inhibitor of an immune mediator selected from the group consisting of soluble interleukin-2 receptor (sIL-2R), soluble interleukin-1 receptor (sIL-1R) and soluble interferon-gamma receptor (sIFN-gammaR) in an immune adsorption element. It is well established that TNF-R1 and TNF-R2 are shed by tumor cells, and that these molecules appear to inhibit immune mediated attack by the host on the tumor cells. These are removed by the present invention. Selective removal of sIL-2R, sIL-1R and/or sIFN-gammaR can be used to supplement the present methods. The later can be removed by binding to a binding gent including the cytokine, mutant, fragment or epitope thereof, or an antibody to the receptor. The above comment and details given with respect to the biding gent for TNF-R1 and TNF-R2 apply as well. The binding agents can be immobilized in the filter, in a column, or using other standard techniques for binding reactions to remove proteins from the blood or plasma of a patient. The biologic activity and clinical effectiveness of pro-inflammatory cytokines is augmented by removal in the patient with cancer. Monocyte and lymphocyte activation is augmented by INF-alpha, INF-beta and gamma.

Highly preferred clinical goals for TNF-R plasma concentrations are in the low normal level ranges for TNF-Rs, especially at most 750 pg/ml for sTNF-R1 and 1250 pg/ml for sTNF-R2 in the plasma, especially to less than 500 pg/ml for sTNF-R1 and 1000 pg/ml for sTNF-R. Accordingly, in another preferred embodiment, the plasma levels are reduced to less than 750 pg/ml for sTNF-R1 and 1250 pg/ml for sTNF-R2 in the plasma, especially to less than 500 pg/ml for sTNF-R1 and 1000 pg/ml for sTNF-R.

In a preferred embodiment, the activated clotting time (ACT) of a blood sample of the patient is controlled during the extracorporeal blood purification process to be kept between 250 and 350 seconds. The activated clotting time (ACT) is commonly used to monitor treatment before, during, and shortly after medical procedures that require that blood be prevented from clotting, such as heart bypass surgery, cardiac angioplasty, thrombolysis, and continuous dialysis. It measures the seconds needed for whole blood to clot upon exposure to an activator of an intrinsic pathway by the addition of e.g. factor XII activators or contact to an artificial surface, such as a cuvette. Cancer patient have a higher risk of blood clots and clotting disturbances. These may be caused by the cancer or the treatment, such as chemotherapy, surgery, medications called steroids, and the long-term use of a catheter. If the ACT should be to low, an inhibitor of blood coagulation (anticoagulant) may be administered to the patient. Suitable and commonly used inhibitors include heparin or citrate. The ACT is usually controlled repeatedly during the extracorporeal blood purification process. The control interval is in the range of from 15 to 6 min. typically, the ACT may be determined every 20 min, every 30 min, every 45 min or every 60 min and an anticoagulant administered, if required.

The cancer to be treated maybe any cancer. Tumor specific inflammation has been observed in patients with many types of cancer including metastatic colon cancer, ovarian cancer, lung cancer, head and neck cancer, cervical and endometrial cancers. In some cases, this inflammation has been followed by significant tumor regressions in each tumor type. Preferably, the cancer is a solid cancer, particularly a solid metastatic cancer. Alternatively or additionally, the cancer is selected from the group consisting of brain cancer, breast cancer, prostate cancer, colon cancer, endometriosis, lung cancer, ovarian cancer, uterine cancer, cervical cancer, melanoma, sarcoma, esophageal cancer, stomach cancer, pancreatic cancer, renal carcinoma, squamous cell of the head and neck and a primary brain malignancy, preferably breast cancer, prostate cancer, and malignant melanoma.

In addition to the present treatment, it is possible to treat the patient with adjuvant or combination therapies that enhance the results achieved with the present treatment. TNF receptors are thought to be particularly important immune inhibitors. Therefore, compounds which enhance TNF activity are particularly preferred. These include anti-angiogenic compounds, such as thalidomide, procoagulant compounds, cytokines and other immunostimulants, such as TNF, interferon-gamma, other interferons, or IL-2, or a procoagulant compound. The treatment increases the inflammation against tumors by allowing cytokines, such as TNF, to work effectively. This provides a basis for an improved effect when combined with any treatment that enhances cytokine activity against the tumors, for example, treatments using alkylating agents, doxyrubicin, carboplatinum, cisplatinum, and taxol, and other drugs which may be synergistic in effect with “unblocked” cytokines. In one preferred embodiment, the selective removal of inhibitors is combined with an immunostimulant, such as a vaccine against tumor antigens, a cytokine to stimulate the immune system or activate dendritic cells, or compounds that block factors such as fibroblast derived growth factor (FDGF), TGF beta, or EGRF. Immune system activation can also be achieved by selective removal of IL-4 and/or IL-10 to drive the cellular mechanism. Standard chemotherapeutic agents, hyperthermia, and/or radiation can also be used with the present treatment.

In the following further specific embodiments are described:
1. A method of treating cancer in a patient, the method comprising a blood purification process comprising
   a) including an extracorporeal circuit comprising a blood-fractionating device having a plasma-separation element and an immune adsorption element into the patient's blood circle;
   b) passing the patient's blood through the extracorporeal circuit, wherein
      (i) blood is continuously taken from the patient;
      (ii) the blood is separated into blood plasma and remainder, wherein blood is passed through the plasma-separation element with a flow rate of at least 300 cm³/min;
      (iii) the blood plasma is purified from soluble TNF receptor-1 (sTNF-R1) and soluble TNF receptor-2 (sTNF-2) in the immune adsorption element, wherein a plasma volume corresponding to at least 20% of the patient's weight is passed through the immune adsorption element per day;
      (iv) blood is restored by combining the purified blood plasma and the remainder; and
      (v) the restored blood is continuously returned into the patient; and
   c) separating the extracorporeal circuit from the patient's blood circle, wherein the blood purification process is performed on at least six days within a period of at most four weeks.
2. The method of embodiment 1, wherein the blood purification process is performed once, twice, three times or four times a day, until the intended daily plasma volume has been purified by passing it through the immune adsorption element.
3. The method of embodiment 1 or 2, wherein the blood purification process is performed on at least 6 days, preferably at least 8 days, more preferably at least 10 days, within a period of at most 21 weeks, preferably within a period of at most 15 days.
4. The method of any of embodiments 1 to 3, wherein the method is continued for at least one month, at least six weeks, at least two months or at least three months.
5. The method of any of embodiments 1 to 4, wherein a plasma volume corresponding to at least 25%, preferably 30%, of the patient's weight is passed through the immune adsorption element per day.
6. The method of any of embodiments 1 to 5, wherein at least 30 l, preferably at least 40 l, more preferably at least 50 l, of the patient's blood are passed through the extracorporeal circuit per day.
7. The method of any of embodiments 1 to 6, wherein blood is passed through the plasma-separation element with a flow rate of at least 350 cm³/min.
8. The method of any of embodiments 1 to 7, wherein the immune adsorption element comprises a column, on which binding agents for sTNF-R1 and sTNF-R2 are covalently bound.
9. The method of any of embodiments 1 to 8, wherein the blood plasma is further purified from at least one inhibitor of an immune mediator selected from the group consisting of soluble interleukin-2 receptor (sIL-2R), soluble interleukin-1 receptor (sIL-1R) and soluble interferon-gamma receptor (sIFN-gammaR) in an immune adsorption element.
10. The method of any of embodiments 1 to 9, wherein the levels are reduced to less than 750 pg/ml for sTNF-R1 and 1250 pg/ml for sTNF-R2 in the plasma, especially to less than 500 pg/ml for sTNF-R1 and 1000 pg/ml for sTNF-R.
11. The method of any of embodiments 1 to 10, wherein the activated clotting time (ACT) of a blood sample of the patient is controlled during the extracorporeal blood purification process to be kept between 250 and 350 seconds, preferably wherein the ACT controlled repeatedly during the extracorporeal blood purification process, more preferably wherein the control interval is in the range of from 15 to 60 min, such as every 20 min, every 30 min, every 45 min or every 60 min and an anticoagulant administered, if required.
12. The method of any of embodiments 1 to 11, wherein the cancer is a solid cancer, particularly a solid metastatic cancer.
13. The method of any of embodiments 1 to 12, wherein the cancer is selected from the group consisting of brain cancer, breast cancer, prostate cancer, colon cancer, endometriosis, lung cancer, ovarian cancer, uterine cancer, cervical cancer, melanoma, sarcoma, esophageal cancer, stomach cancer, pancreatic cancer, renal carcinoma, squamous cell of the head and neck and a primary brain malignancy, preferably breast cancer, prostate cancer, and malignant melanoma.

The invention is not limited to the particular methodology, protocols and reagents described herein because they may vary. Furthermore, the terminology used herein is for the purpose of describing particular embodiments only and is not intended to limit the scope of the present invention. As used herein and in the appended claims, the singular forms “a”, “an”, and “the” include plural reference unless the context clearly dictates otherwise. Similarly, the words “comprise”, “contain” and “encompass” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, and materials are described herein.

The present invention is further illustrated by the following Examples, from which further features, embodiments and advantages may be taken. As such, the specific modifications discussed are not to be construed as limitations on the scope of the invention. It will be apparent to the person skilled in the art that various equivalents, changes, and modifications may be made without departing from the scope of the invention, and it is thus to be understood that such equivalent embodiments are to be included herein.

### EXAMPLES

### Example 1: Exemplary Treatment Protocol

In the following an exemplary treatment protocol is provided which may be used for the blood purification process of the present invention. It is evident that each of the below steps can be combined with the method of the invention, as described in the claims and the description:

| | |
|---|---|
| Step 1 | Before the treatment can be applied the patient has to receive a dialysis |
| | catheter that allows flow rates of 350 ml/min which has to be |
| | subcutaneously tunneled. The recommended type is Gambro |
| | RetroPouchez (Modell Retro Pourchez; Gambro). |
| Step 2 | Ensure continuous aseptic catheter care until removal of the catheter. |
| Step 3 | Equip LentzLoc approved apheresis machine (Bavarian Immunology |
| | Association GmbH, Prien, Germany; see the manufacturer's instructions |
| | for use) with the tubings, filters and other required material according to |
| | manufacturer's instructions (use aseptic technique). |
| Step 4 | Prime the system according to the manufacturer's instructions. |
| Step 5 | Rinse LentzLoc Immunoadsorption Column (Bavarian Immunology |
| | Association GmbH, Prien, Germany) with normal saline according to |
| | manufacturer's instructions (use aseptic technique). |
| Step 6 | Install rinsed LentzLoc column in the primed machine and connect it |
| | according to the manufacturer's instructions (use aseptic technique). |
| Step 7 | Examine patient and document vital signs and patient's weight and any |
| | physical complaints that they have. |
| Step 8 | Prepare patient's dialysis catheter for use (use aseptic technique). |
| | Make sure that the feasible blood flow rate is at least 300 ml/min. Take |
| | adequate action to ensure. |
| Step 9 | Take blood samples (use aseptic technique): Make complete blood |
| | count (CBC) plus differential, chemistries and blood tumor markers, if |
| | indicated, at least once weekly, additionally as ordered by physician. |
| Step 10 | Connect dialysis catheter to blood tubing set of the system (use aseptic |
| | technique). |
| Step 11 | Begin the LentzLoc adsorption process according to the manufacturer's |
| | instructions. |
| Step 12 | Set blood flow rate at 250 ml/min and increase to 300 ml/min (minimum) |
| | within the next 10 minutes. |
| | Set plasma flow rate at 5% of blood flow rate and increase to 30% over |
| | the next 30 minutes. |
| Step 13 | Determine activated clotting time (ACT) of a blood sample of the |
| | patient. It should be in the range of from 250 to 350 seconds. If the |
| | Volume is to low, administer to the patient's tubing system heparin or |
| | citrate, as appropriate. Control ACT every 30 minutes and adjust to |
| | maintain 250-350 s. |
| Step 14 | If during the procedure the patient develops tumor specific pain, poorly |
| | controlled with i.v. narcotics or temperature over 38.5° C. temperature or |
| | hyper- or hypotension, allergic symptoms, acute neurologic changes |
| | discontinue the procedure. In case of elevated temperature the patient |
| | can continue if the physician allows. |
| Step 15 | Set the daily plasma volume to be treated between 20-30% of patient's |
| | weight so as to decrease TNF-inhibitors to R1 to 500 pg/ml, to R2 to |
| | 1000 pg/ml. Adjust subsequent ultrafiltrate volume accordingly to |
| | achieve this goal. |
| Step 16 | After 7 liters of ultrafiltrated plasma are treated proceed to the |
| | recirculation mode. Follow the instructions of the manufacturer. |
| | Return blood as per manufacturer's recommendations; disconnect the |
| | patient according to manufacturer's instruction (use aseptic technique). |
| Step 17 | Heparinize the remaining blood in the apheresis tubing set, to avoid |
| | clotting during the break (use aseptic technique). |
| Step 18 | Prepare catheter blocking solution (heparin (1.000 IE/ml); 0.72% NaCl) |
| | for the break (use aseptic technique) |
| | Administer to individual catheter lumen volume. |
| Step 19 | Collect ultrafiltrate samples at least on day 1, 3, 5, 9, 12 and 15 and |
| | other days as recommended and required by the attending physician. |
| | Ultrafiltrate samples (1-2 ml) are collected through a 3-way-stop cock |
| | before the LentzLoc column at 0.5 l and end of treatment of plasma |
| | volume run through the LentzLoc column (use aseptic technique). |
| Step 20 | Regenerate column as per manufacturer's instructions (use aseptic |
| | technique). |
| | After 20-40 minute treatment break for about 25 to 30 min. |
| | Reconnection of column and patient to the machine and proceed with |
| | another 7 liters of treated ultrafiltrate until the treatment goal (see step |
| | 15) is achieved. |
| Step 21 | At the end of the prescribed treatment of ultrafiltrated plasma return the |
| | blood to the patient according to manufacturer's instruction (use aseptic |
| | technique). |
| Step 22 | Administer proper catheter care as the patient is disconnected from the |
| | machine (use aseptic technique). |
| Step 23 | Redress the catheter site; fill each catheter end tube with the adequate |
| | amount of catheter lock solution (Taurolock) from TauroPharm. The |
| | right volume of the lock solution can be found on each individual |
| | catheter. |
| | Take vital signs and weight (use aseptic technique). |
| | Observe the patient for the next 30 minutes. |
| Step 24 | Remove tubing sets, filter from the machine and the column according |
| | to manufacturers' recommendations, and discard the tubing sets and |
| | the filter as biologic waste. |
| Step 25 | Clean the machine. Wipe the machine down with appropriate antiseptic. |
| Step 26 | End-of-day regeneration of the LentzLoc column according to the |
| | manufacturer's recommendation (use aseptic technique). |
| Step 27 | Store the LentzLoc column in a temperature controlled fridge at 2-8° C. |
| | till next use. (Do not freeze). If out of this temperature range discard the |
| | LentzLoc column. |
| Step 28 | Have maximum 15 consecutive treatments with one LentzLoc column, |
| | then discard the column with the biologic waste. |

### Example 2: Clinical Assessment of Treatments

Patients treated almost all suffer from late-stage metastatic cancer. Most of these patients have already failed standard of care and are immune compromised as a result. One would expect little to no clinical improvement in response to any treatment modality with patients as sick as these.

It is thus all the more remarkable that for the 10 cancer types that respond best† to the treatment in question, the Overall Response Rate (ORR) is 75.4% (average N per cancer type=6.9). “Overall Responses” consist exclusively of Complete Responses (CR)—i.e. remission—and Partial Responses (PR)—meaning a decrease of tumor diameters of 50% or more, which is clinically very significant. Of the above Overall Responses, 42.3% were CRs and 57.7% were PRs. For the top five cancer types†† that respond best to treatment, the ORR is 100%. Considering all 102 evaluable patients treated—representing 25+ solid tumor cancer types—Overall Responses have been documented in 61 cases (60%).

The leading theory as to why some patients with certain metastatic cancer types respond better than others is that the more responsive patients have generally received less immuno-suppressive treatment—especially chemotherapy—before reaching the treatment. However, even these lower observed responses significantly exceed what would be expected with other treatment modalities. The expected ORR to third and fourth line chemotherapies is <10% for breast and ovarian cancer, and <3% for soft tissue sarcomas, brain, melanoma, lung, colo-rectal and prostate cancers. In addition, most patients report that treatment is significantly easier to withstand than chemotherapy and/or radiation.

The table below presents specific response rates for the various cancer types. While response rates for the treatment according to the present invention are higher than for any other treatment modality, it's important to be aware of two key caveats:

1) Recurrence is Common without Maintenance Treatments.

While a surprising number of patients treated reach full remission (CR)—defined as the absence of detectable disease—many subsequently undergo recurrence unless they undergo regular maintenance treatments. The interval before recurrence without maintenance treatments varies from a few months to several years or more, with 6-18 months being the average. The International Immunology Foundation is currently researching ways of extending the period of remission, with promising results. Fortunately, unlike chemotherapy, the treatment of the present invention is generally effective in repeat applications. For many patients, this is a maintenance program, not a cure.

2) CBR does not Equal Remission.

One of the data points presented below is “Combined Benefit Rate.” CBR is the total percentage of all patients who respond positively to treatment. A positive response is anything other than disease progression—everything from full remission to stable disease (i.e. not getting worse). This calculation is included because it's popular with drug companies, given that it captures all efficacy no matter how slight and thus presents a treatment in the best possible light. For many cancer types, the present treatment has documented CBRs of 100%—meaning that not a single patient with that cancer type got worse while undergoing treatment. For most patients, Overall Response Rates are more meaningful.

| Cancer Type | Overall Response Rate (CR + PR) | | | CBR |
|---|---|---|---|---|
| [all late stage metastatic] | # of ORs | Total N | % | % |
| Brain | 3 | 3 | 100% | 100% |
| Breast | 20 | 27 | 74% | 100% |
| Endometrial | 1 | 1 | 100% | 100% |
| Lung (various) | 6 | 13 | 46% | 100% |
| Melanoma | 2 | 3 | 67% | 100% |
| Ovarian | 3 | 4 | 75% | 100% |
| Prostate | 8 | 8 | 100% | 100% |
| Renal | 1 | 1 | 100% | 100% |
| Sarcoma/soft-tissue (various) | 6 | 7 | 86% | 100% |
| Sq-cell of head & neck | 2 | 2 | 100% | 100% |

These data suggest that response rates to the present treatment are indeed significant, but the N's for some cancer types might be too small to draw firm conclusions, and further research is therefore indicated. Additional trials are currently being planned to determine precise response rates by tumor type; time to disease progression (with and without regular maintenance treatments); duration of survival and quality of life while on treatment.

### Definitions

CR—Complete Response: Disappearance of all measurable disease, i.e. “remission”
PR—Partial Response: Decrease in diameters of tumors by 50% or more *
MR—Minimal Response: Decrease in diameters of tumors by 25-49%
SD—Stable Disease: No increase—or decrease in diameters of tumors by <25%—while on treatment or 30 days post treatment
PD—Progressive Disease: An increase in diameters of tumors during study or 30 days post treatment
ORR—Overall Response Rate: The sum of the first 2 response rates above, i.e. CR+PR
CBR—Clinical Benefit Rate: The sum of the first 4 response rates above, i.e. CR+PR+MR+SD
All measurements are the sum of products of greatest diameters of measurable tumors.

† The 10 metastatic cancer types treated with the highest documented ORR are Brain, Breast, Endometrial, Lung, Melanoma, Ovarian, Prostate, Renal, Soft-tissue Sarcomas, and Squamous cell of the head & neck. (Brain cancer—consisting of Astrocytoma 11 & Glioblastoma Multiforme—is included on this list although not metastatic.)

†† The top five responding metastatic cancer types—each with an ORR of 100%—are Brain, Endometrial, Prostate, Renal, and Squamous cell of the head & neck.

1. A method of treating cancer in a patient, the method comprising a blood purification process comprising
a) including an extracorporeal circuit comprising a blood-fractionating device having a plasma-separation element and an immune adsorption element into the patient's blood circle;
b) passing the patient's blood through the extracorporeal circuit, wherein
(i) blood is continuously taken from the patient;
(ii) the blood is separated into blood plasma and remainder, wherein blood is passed through the plasma-separation element with a flow rate of at least 300 cm³/min;
(iii) the blood plasma is purified from soluble TNF receptor-1 (sTNF-R1) and soluble TNF receptor-2 (sTNF-2) in the immune adsorption element, wherein a plasma volume corresponding to at least 20% of the patient's weight is passed through the immune adsorption element per day;
(iv) blood is restored by combining the purified blood plasma and the remainder; and
(v) the restored blood is continuously returned into the patient; and
c) separating the extracorporeal circuit from the patient's blood circle,
wherein the blood purification process is performed on at least six days within a period of at most four weeks.

2. The method of claim 1, wherein the blood purification process is performed once, twice, three times or four times a day, until the intended daily plasma volume has been purified by passing it through the immune adsorption element.

3. The method of claim 1, wherein the blood purification process is performed on at least 6 days within a period of at most 15 days.

4. The method of claim 1, wherein the method is continued for at least one month.

5. The method of claim 1, wherein a plasma volume corresponding to at least 25 of the patient's weight is passed through the immune adsorption element per day.

6. The method of claim 1, wherein at least 30 l of the patient's blood are passed through the extracorporeal circuit per day.

7. The method of claim 1, wherein blood is passed through the plasma-separation element with a flow rate of at least 350 cm³/min.

8. The method of claim 1, wherein the immune adsorption element comprises a column, on which binding agents for sTNF-R1 and sTNF-R2 are covalently bound.

9. The method of claim 1, wherein the blood plasma is further purified from at least one inhibitor of an immune mediator selected from the group consisting of soluble interleukin-2 receptor (sIL-2R), soluble interleukin-1 receptor (sIL-1R) and soluble interferon-gamma receptor (sIFN-gammaR) in an immune adsorption element.

10. The method of claim 1, wherein the levels are reduced to less than 750 pg/ml for sTNF-R1 and 1250 pg/ml for sTNF-R2 in the plasma.

11. The method of claim 1, wherein the activated clotting time (ACT) of a blood sample of the patient is controlled during the extracorporeal blood purification process to be kept between 250 and 350 seconds and an anticoagulant administered, if required.

12. The method of claim 1, wherein the cancer is a solid cancer.

13. The method of claim 1, wherein the cancer is selected from the group consisting of brain cancer, breast cancer, prostate cancer, colon cancer, endometriosis, lung cancer, ovarian cancer, uterine cancer, cervical cancer, melanoma, sarcoma, esophageal cancer, stomach cancer, pancreatic cancer, renal carcinoma, squamous cell of the head and neck and a primary brain malignancy.
